(19) United States
(12) Patent Application Publication
(10) Pub. No.: US 2020/0043577 A1
(43) Pub. Date: Feb. 6, 2020

(54) DEVICE AND METHOD FOR ESTABLISHING ELECTRONIC MEDICAL RECORD

(71) Applicant: Jiang Co., Ltd., Hsinchu City (TW)

(72) Inventor: Jeng-Jier JIANG, Hsinchu City (TW)

(21) Appl. No.: 16/458,650

(22) Filed: Jul. 1, 2019

Related U.S. Application Data
(60) Provisional application No. 62/714,318, filed on Aug. 3, 2018.

(30) Foreign Application Priority Data
Sep. 26, 2018 (TW) 107133888

(51) Int. Cl. G16H 10/60; G06F 16/51; G06F 16/583

(52) U.S. Cl. CPC G16H 10/60; G06F 16/5846; G06F 16/51

(57) ABSTRACT

A method of establishing an electronic medical record includes steps of converting a paper medical record into an electronic image, saving the electronic image in a database, and retrieving the electronic image by a first handheld device to create an electronic medical record image having a layer thereon, embedding medical information into the electronic medical record image via external input, and saving the electronic medical record image back to the database.

### CROSS REFERENCE TO RELATED APPLICATIONS

This application claims the benefit of the filing date of U.S. provisional patent application Ser. No. 62/714,318, filed on Aug. 3, 2018, and the priority of Taiwan Patent Application No. 107133888, filed on Sep. 26, 2018, the disclosures of which are incorporated herein by reference in their entirety.

### FIELD OF INVENTION

The present disclosure relates to a device and a method for establishing an electronic medical record, and more particularly to a device and a method for establishing an electronic medical record, which is capable of converting a traditional paper medical record into an electronic medical record, and capable of directly writing, drawing, and pasting various types of digital media files onto the electronic medical record.

### BACKGROUND OF INVENTION

Owing to ongoing technological advancement, traditional paper medical records have been gradually replaced by electronic medical records. Electronic medical records enable medical personnel to quickly access medical records, which saves time for searching medical records and delivering medical records. Electronic medical records also overcome problems of the paper medical records that occupy a huge space and are difficult to preserve. Therefore, the important medical records of each patient can be preserved for a long time.

However, traditional electronic medical record systems employ standardized interfaces. Medical personnel have to learn function menus and manuals of each medical record system again. Furthermore, medical personnel can only fill out patients' information in preset text fields which are provided in the interface. For the diseases that patients have, because each type of diseases shares one diagnosis code, patients are automatically filled with the same content. Therefore, traditional electronic medical record systems cannot accurately describe every patient's condition and also does not allow to draw pictures to record the patient's condition, which is unfavorable to follow-up tracking according to each patient's different conditions or is unfavorable to perform reliable medical statistics on the type of diseases to promote medical progresses.

Furthermore, traditional electronic medical records are stored as digital bytes. Even if the traditional electronic medical records are combined with an electronic signature, it is still impossible to distinguish the identity of the user is the physician himself or not. Therefore, a person, who has bad intentions, can violate regulations using this loophole, such as committing an impersonation.

### SUMMARY OF INVENTION

A primary object of the present disclosure is to provide a device and a method for establishing an electronic medical record, so that medical personnel can directly record, draw, and even paste various types of digital media files onto an electronic medical record by a handheld device, which is not limited by standardized menus and text fields that are provided by the traditional electronic medical record system. Each patient's condition can be accurately recorded to facilitate tracking of the disease.

A secondary object of the present disclosure is to provide a device and a method for establishing an electronic medical record to directly read the text that is handwritten by medical personnel on an electronic medical record so that the handwriting of the medical personnel can be preserved in the electronic medical record. Therefore, each electronic medical record can be traced, thereby solving the problem of the traditional electronic medical record system that it is still impossible to distinguish the identity of the user, and avoiding the problem of the traditional electronic medical record system that a person who has bad intentions can commit an impersonation using this loophole. Furthermore, the handwritten text on the electronic medical record of the present disclosure is machine-readable and can be used to perform functions, such as searching. Credibility and practicability of the electronic medical record are improved at the same time.

Another object of the present disclosure is to provide a device and a method for establishing an electronic medical record to directly capture an electronic image of a paper medical record to create an electronic medical record image having a layer thereon, such that the electronic medical record image serves as a virtual paper and allows the medical personnel to write directly on the electronic medical record image. Therefore, an establishment of the electronic medical record is achieved without learning a manual of a new electronic medical record system, thereby improving overall medical efficiency.

In order to achieve the above objects, the present disclosure provides a method for establishing an electronic medical record, comprising steps of:

converting a paper medical record into an electronic image;

saving the electronic image in a database;

retrieving the electronic image by a first handheld device from the database to create an electronic medical record image having a layer thereon, and embedding medical information into the electronic medical record image via external input, wherein the external input is a text, a handwriting input, a picture, or a digital media file; and

saving the electronic medical record image back to the database.

According to an embodiment of the present disclosure, the database is located in a server.

According to an embodiment of the present disclosure, the database is located in a storage device of the first handheld device.

According to an embodiment of the present disclosure, the method further comprises adding another layer on the layer of the electronic medical record image by the user.

According to an embodiment of the present disclosure, the method further comprises recognizing the text when the text is handwritten on the electronic medical record image, such that the first handheld device can directly read the text that is handwritten on the electronic medical record image.

According to an embodiment of the present disclosure, the method further comprises conducting a search according to the text in the electronic medical record image by a search criterion and displaying a searched result in a list in a sorted order.

According to an embodiment of the present disclosure, the method further comprises saving an original content of the electronic medical record image while modifying medical information thereof.

According to an embodiment of the present disclosure, the method further comprises establishing a mobile medical record database, establishing a plurality of classifications in the mobile medical record database, classifying the electronic medical record image, and placing the electronic medical record image in one of the classifications of the mobile medical record database according to an order of classification.

According to an embodiment of the present disclosure, the method further comprises converting the electronic medical record image into a signable file and embedding a handwritten signature or a stamp image on an arbitrary position on the electronic medical record image.

According to an embodiment of the present disclosure, the method further comprises encrypting the electronic medical record image and accessing the electronic medical record image according to an authorization of the user.

According to an embodiment of the present disclosure, the method further comprises encrypting the electronic medical record image, transmitting the encrypted file to a second handheld device, decrypting the encrypted file by the second handheld device, and reading the electronic medical record image by the second handheld device from the decrypted file.

According to an embodiment of the present disclosure, the method further comprises playing the digital media file on the electronic medical record image.

The present disclosure further provides a device for establishing an electronic medical record, comprising:

an image capturing device, wherein the image capturing device is configured to convert a paper medical record into an electronic image;

a touch panel, wherein the touch panel is configured to receive a touch signal and a data input signal, and to display the electronic image;

an electronic medical record system, wherein the electronic medical record system is configured to retrieve the electronic image from the database to create an electronic medical record image having a layer thereon and is configured to embed medical information into the electronic medical record image via external input, wherein the external input is a text, a handwriting input, a picture, or a digital media file;

a storage device for saving the electronic medical record image; and

a network communication device connected to a server for uploading the electronic medical record image to the server and accessing the electronic medical record image in the server.

According to an embodiment of the present disclosure, the server comprises:

a template database having an electronic image of a paper medical record template, wherein the electronic medical record system accesses the paper medical record template from the template database via the network communication device; and

a medical record database having the electronic medical record image, wherein the medical record database is connected to the storage device via the network communication device and stores the electronic medical record image that is saved in the storage device.

According to an embodiment of the present disclosure, the device further comprises a template database having an electronic image of a paper medical record template.

According to an embodiment of the present disclosure, the electronic medical record system comprises an encryption module for encrypting the electronic medical record image, whereby the user can access the electronic medical record image according to an authorization of the user.

According to an embodiment of the present disclosure, the electronic medical record system comprises an analysis module, wherein the analysis module is configured to recognize the text when the text is handwritten on the electronic medical record image, such that the electronic medical record system can directly read the text that is handwritten on the electronic medical record image.

According to an embodiment of the present disclosure, the electronic medical record system comprises a search module for conducting a search according to the text in the electronic medical record image by a search criterion and displaying a searched result in a list in a sorted order.

According to an embodiment of the present disclosure, the electronic medical record system comprises a mobile medical record database, wherein the mobile medical record database comprising a plurality of classifications and the electronic medical record image is placed in one of the classifications of the mobile medical record database according to a sorting criterion.

The present disclosure provides a device and a method for establishing an electronic medical record, so that medical personnel can directly record, draw, and even paste various types of digital media files onto an electronic medical record by a handheld device, which is not limited by standardized menus and text fields that are provided by the traditional electronic medical record system. Each patient's condition can be accurately recorded to facilitate tracking of the disease.

The device for establishing the electronic medical record directly read the text that is handwritten by medical personnel on an electronic medical record, so that the handwriting of the medical personnel can be preserved in the electronic medical record. Therefore, each electronic medical record can be traced, thereby solving the problem of the traditional electronic medical record system that it is still impossible to distinguish the identity of the user, and avoiding the problem of the traditional electronic medical record system that a person who has bad intentions can commit an impersonation using this loophole. The patient's rights and interests are thus protected. Furthermore, the handwritten text on the electronic medical record of the present disclosure is machine-readable and can be used to perform functions, such as searching. Credibility and practicability of the electronic medical record are improved at the same time.

The device for establishing an electronic medical record directly capture an electronic image of a paper medical record to create an electronic medical record image having a layer thereon, such that the electronic medical record image serves as a virtual paper and allows the medical personnel to write directly on the electronic medical record image. Therefore, an establishment of the electronic medical record is achieved without learning a manual of a new electronic medical record system, thereby improving overall medical efficiency.

Other features and embodiments of the present disclosure will be further understood from the following detailed description in combination with the drawings.

### DESCRIPTION OF DRAWINGS

FIG. 1 is a schematic diagram of a device and a server for establishing an electronic medical record system according to an embodiment of the present disclosure.

FIG. 2 is a block diagram of modules of the device for establishing an electronic medical record according to an embodiment of the present disclosure.

FIG. 3 is a structure chart of a mobile medical record database according to an embodiment of the present disclosure.

FIG. 4 is a flow chart of a method for establishing an electronic medical record according to an embodiment of the present disclosure.

FIG. 5 is a flow chart of another method of establishing an electronic medical record according to an embodiment of the present disclosure.

FIG. 6 and FIG. 7 are perspective views of a device for establishing an electronic medical record from different angles according to an embodiment of the present disclosure.

REFERENCE NUMERALS IN DRAWINGS

| | |
|---|---|
| 10 a device of establishing an electronic medical record; | 101 a touch panel; |
| 102 an image capture device; | 103 an electronic medical record image; |
| 104 a stylus; | 105 a first button; |
| 106 a second button; | 107 a storage device; |
| 108 a network communication device; | 109 a mobile medical record database; |
| 11 a second handheld device; | 12 a first handheld device; |
| 13 a third handheld device; | 14 a fourth handheld device; |
| 15 a patient; | 16 a nurse; |
| 17 a physician; | 18 a pharmacist; |
| 19 network communication; | 20 a server; |
| 201 a template database; | 202 a medical record database; |
| 203 a data processor; | 204 an identification module; |
| 205 a data encryption module; | 31 a paper medical record; |
| 32 an electronic image; | 33 a paper medical record template; |
| 34 an electronic medical record; | 40 an electronic medical record system; |
| 401 a handwriting module; | 402 an encryption module; |
| 403 an analysis module; | 404 a search module; |
| 50 an input text; | 51 a handwritten text; |
| 52 a picture; | 53 a digital media file; |
| 54 a stamp; | 55 a signature; |
| 56 a layer; | 60 a consent form; |
| 601 a donation willingness form; | 61 today's appointments; |
| 611 morning appointments; | 612 afternoon appointments; |
| 613 night appointments; | 62 first-time medical records; |
| 63 a medical record library; | 631 general; |
| 632 severe; | 633 postpartum confinement; |
| 634 assistance; | 64 babies; |
| 641 postpartum confinement; | 642 babies from wards; |
| 65 reports; | 651 examination reports; |
| 652 pathological reports; and | 66 others. |

### DETAILED DESCRIPTION OF PREFERRED EMBODIMENTS

The above objects, features, and advantages of the present disclosure herein become more readily apparent upon reference to specific embodiments of the present disclosure described in detail with accompanying drawings. In this regard, directional terminology mentioned in the present disclosure, such as “up”, “down”, “top”, “bottom”, “front”, “back”, “left”, “right”, “inner”, “outer”, “lateral”, “around”, “central”, “horizontal”, “transversal”, “vertical”, “longitudinal”, “axial”, “radial”, “uppermost”, “lowermost”, etc., is used with reference to the orientation of the figures being described. Therefore, the directional terminology is used for purposes of illustration and is not intended to limit the present invention.

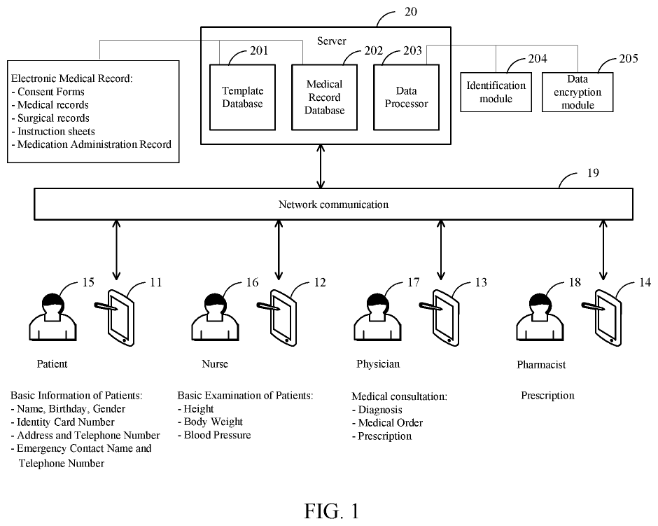

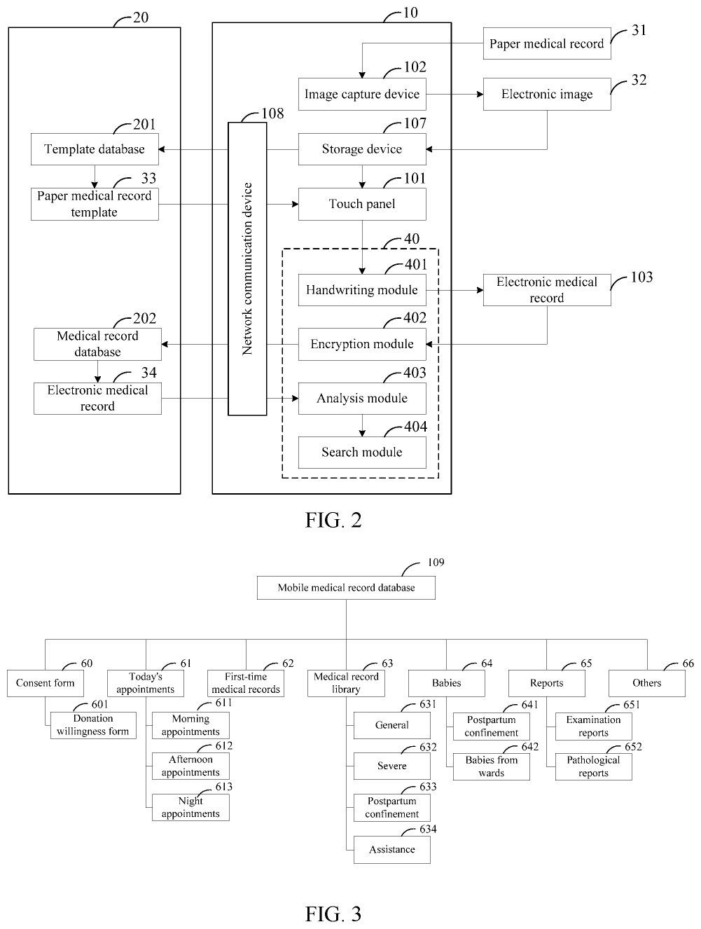

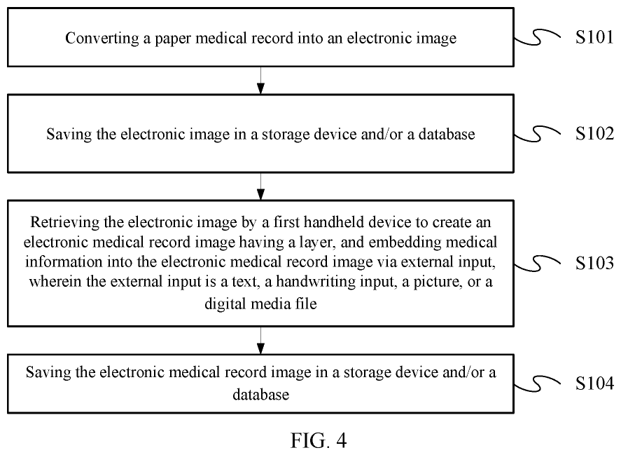

Please refer to FIG. 1 to FIG. 3. FIG. 1 is a schematic diagram of a device and a server for establishing an electronic medical record system according to an embodiment of the present disclosure. FIG. 2 is a block diagram of modules of the device for establishing an electronic medical record according to an embodiment of the present disclosure. FIG. 3 is a structure chart of a mobile medical record database according to an embodiment of the present disclosure. A device 10-14 for establishing an electronic medical record of the present disclosure can connect to a server 20 via a network communication 19 and thus connect to each other. The server 20 includes a template database 201, a medical record database 202, and a data processor 203. The device 11-14 can be selected from one of a tablet, a mobile phone, and a tablet-laptop hybrid.

As shown in FIG. 2, a device 10 for establishing an electronic medical record according to an embodiment of the present disclosure includes an image capturing device 102, a touch panel 101, an electronic medical record system 40, a storage device 107, and a network communication device 108. The image capture device 102 converts a paper medical record 31 into an electronic image 32. In an embodiment of the present disclosure, the paper medical record 31 can have one or more than one page and each page of the paper medical record 31 is converted into an electronic image 32. The paper medical record 31 can be a blank paper medical record so as to create a paper medical record template 33. The paper medical record 31 can also be a documented paper medical record so as to convert the documented paper medical record into an electronic medical record 34. The image capturing device 102 can be a camera or a scanner. If the image capturing device 102 is a camera of the device 10, the device 10 is directly used to photograph the paper medical record 31. If the image capturing device 102 is a scanner, the paper medical record 31 is placed in the scanner to preform scanning. The touch panel 101 is configured to receive a touch signal and a data input signal, and display the electronic image 32. The touch panel 101 receives a touch signal that is applied on the touch panel 101 by a user to control the electronic medical record system 40. The touch panel 101 displays a corresponding image after receiving the data input signal. For example, when the touch panel 101 receives a data input signal of the electronic image 32, the touch panel 101 displays the electronic image 32.

The storage device 107 is configured to save the edited electronic medical record 34 and/or the electronic medical record image 103. The storage device 107 can receive the electronic medical record 34 from the server 20 and store the electronic medical record 34 in the storage device 107. The network communication device 108 is connected to a server 20. The network communication device 108 can upload the edited electronic medical record 34 and/or the electronic medical record image 103 to the server 20, and retrieve the edited electronic medical record 34 and/or the electronic medical record image 103 from the server 20 by WIFI system, BLUETOOTH, mobile ad hoc network (MANET), airdrop, near-field communication (NFC), mobile communication technologies (for example, 3C, 4C, 5C, etc.), global system for mobile communications (GSM), general packet radio service (GPRS), personal handy-phone system (PHS), wireless local area networking (WLAN), or worldwide interoperability for microwave access (WiMAX).

The template database 201 includes at least one paper medical record template 33, for example, a consent template, a medical record template, a surgical record template, an instruction template, a prescription record template, a donation template, a blood drawing record template, a medical examination form template, a birth record template, etc. The paper medical template 33 stored in the template database 201 can be derived from an electronic image 32 captured from a paper medical record template 33 by the image capture device 102.

The data processor 203 includes an identification module 204 and a data encryption module 205. The identification module 204 can administer authorization of the users 15-18 to the server 20. The data encryption module 205 can encrypt the electronic medical record 34 that is stored in the server 20.

The storage device 107 of the device 10 can store at least one electronic image 32 of at least one paper medical record template 33. The storage device 107 of the device 10 can store various types of templates, such as: a consent template, a medical record template, a surgical record template, an instruction template, a prescription record template, a donation template, a blood drawing record template, a medical examination form template, a birth record template, etc. The paper medical template 33 stored in the storage device 107 can be derived from an electronic image 32 captured from a paper medical record template 33 by the image capture device 102.

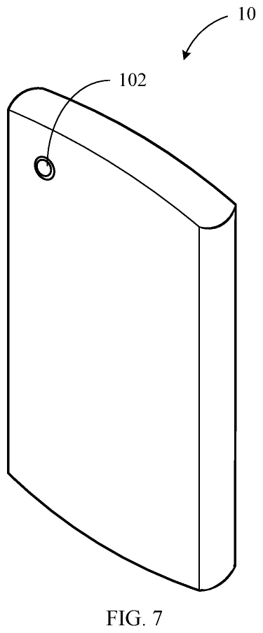

The electronic medical record system 40 can include a handwriting module 401, an encryption module 402, an analysis module 403, and a search module 404. The handwriting module 401 in the electronic medical record system 40 accesses the electronic image 32 and retrieves the electronic image 32 to create an electronic medical record image 103 having a layer 56. Therefore, a user (for example, a patient 15, a nurse 16, a physician 17, a pharmacist 18, etc.) can edit the layer 56 of the electronic medical record image 103 or inputting medical information onto the electronic medical record image 103 on the touch panel 101 by inputting a text (see 50 in FIG. 6), handwriting a text (see 51 in FIG. 6), drawing a picture (see 52 in FIG. 6), and/or pasting a digital media file (see 53 in FIG. 6) on the layer 56 of the electronic medical record image 103 to create an electronic medical record 34. The electronic medical record 34 can have one or more than one page of the electronic medical record image 103. The electronic medical record image 103 can be a medical record, a medical record continuation page, an examination result, a consent form, a prescription, a blood drawing form, a medical examination form, or an arrears form, the handwriting module 401 can be configured to automatically upload the electronic medical record 34 of the electronic medical record 34 and/or the electronic medical record 34 to the server 20 so as to update the electronic medical record 34 of the server 20 in real time.

The electronic medical record system 40 accesses a paper medical record template 33 from the template database 201 of the server 20 via the network communication device 108. A medical record database 202 of the server 20 is connected to the storage device 107 via the network communication device 108. The electronic medical record 34 and/or the electronic medical record image 103 stored in the storage device 107 are saved in the medical record database 202. The electronic medical record image 103 can be a consent form, a medical record, a surgical record, an instruction, a prescription record template, a donation template, a blood drawing form, a medical examination form, a birth record, etc. The electronic medical record 34 stored in the medical record database 202 can be derived from at least one electronic image 32 captured from a paper medical record 31 by the image capture device 102 or can be derived from the electronic medical record 34 established by the device 10. Because the paper medical record 31 can have at least one page, the electronic medical record 34 established by the paper medical record 31 can have at least one electronic medical record image 103.

According to an embodiment of the present disclosure, a first-time patient 15 accesses at least one paper medical record template 33 in the template database 201 via a network communication 19 using a handheld device 11 and writes or input basic personal information, such as name, birthdate, gender, identity card number, address, telephone number, emergency contact name and phone number, blood type, drug allergy record, current medication, disease history, etc. on the paper medical record template 33 to create a first-time medical record 62. The first-time medical record 62 is transmitted to the medical record database 202 via the network communication 19 by the network communication device 108. A nurse 16 accesses the first-time medical record 62 via the network communication 19 by a handheld device 12 and input the results obtained from a basic examination which is performed on the patient 15 onto the first-time medical record 62, such as height, weight, blood pressure, heart rate, body temperature, etc. The first-time medical record 62 is then transmitted to the medical record database 202 via the network communication 19. A physician 17 uses a handheld device 13 to access the first-time medical record via the network communication 19 so as to provide medical consultation to the patient 15 and make a diagnosis, writes a description of a disease, a medical order, and a prescription on the first-time medical record 62. The first-time medical record 62 is then transmitted to the medical record database 202 via the communication network 19. A pharmacist 18 uses a handheld device 14 to access the prescription via the communication network 19 and dispenses medications for the patient 15.

The electronic medical record system 40 can include an encryption module 402. The encryption module 402 is configured to encrypt the electronic medical record image 103. Therefore, the users 15-18 can access the electronic medical record image 103 according to an authorization of the users 15-18. According to an embodiment of the present disclosure, after editing the electronic medical record image 103, the encryption module 402 encrypts the electronic medical record image 103, for example, by generating a code, setting a password lock, setting a fingerprint recognition lock, setting a face recognition lock, etc. for the electronic medical record image 103. The electronic medical record image 103 is then uploaded to the server 20. According to another embodiment of the present disclosure, an encryption module 402 of a handheld device 13 of the physician 17 generates a code for the electronic medical record image 103 and transmits the code to a second handheld device 11 of the patient 15. The second handheld device 11 of the patient 15 decrypts the code and reads the electronic medical record image 103. The code can be selected from one of a one-dimensional code, a two-dimensional code, a quick response matrix code (QR-code), etc. The method and the device 10 for establishing an electronic medical record of the present disclosure can also be used to encrypt a huge amount of data, such as an examination report, a brochure for health education, a mother handbook, and a parent handbook into a code, and transmit the code to a user, for example, a patient 15 or medical personnel 16-18.

The electronic medical record system 40 can include an analysis module 403. The analysis module 403 is configured to recognize the text handwritten on the layer 56 of the electronic medical record image 103 (see 51 of FIG. 6) and estimate a literal meaning of the text 51 (i.e. estimate a machine-readable representation (for example, binary code, ASCII code, Unicode, etc.) corresponding to the character of the handwritten text 51), such that the electronic medical record system 40 can directly read the text 51 that is handwritten on the layer 56 of the electronic medical record image 103 and/or read the representation of the text 51 handwritten on the layer 56 of the electronic medical record image 103. The recognition may be performed by a comparison method. The comparison method may include comparing the handwritten text 51 with a plurality of characters in a character library. The analysis module 403 can compare the text 51 in a process of handwriting a text 51 on a touch panel of the handheld device 11-14 and estimate the corresponding representation of the text 51 to recognize each handwritten text 51 and/or record the representation corresponding to each handwritten text 51. Each handwritten text 51 serves as an image in which each pixel is arranged in a two-dimensional (2D) array. The analysis module can be configured to read rows and columns of the 2D array and convert the rows and the columns into at least an independent matrix for each character. The matrix is then compared with the characters of the character library to estimate the meaning and/or the representation corresponding to the handwritten text 51, so that the text 51 handwritten on the layer 56 of the electronic medical record image 103 are machine-readable and selectable by the electronic medical record system 40 while being shown as a handwriting text. If the text to be compared is a language in which a plurality of letters are concatenated into a single word (for example, English, Latin, German, Italian, etc.), the characters of the character library which the text is compared with are the letters of the language. If the text to be compared is constructed from radicals and components or other forms (for example, Chinese, Korean, etc.), the characters of the character library which the text is compared with are the text itself. If the text to be compared is Arabic numerals, then the characters of the character library which the text is compared are a number. If the text to be compared is a punctuation mark (for example, “,”, “.”, “,”, “.”, etc.), the characters of the character library which the text are compared with is the text itself. The text 51 handwritten on the layer 56 of the electronic medical record image 103 in FIG. 6 is exemplified by an English—‘Disease’. The texts “D”, “i”, “s”, “e”, “a”, “s”, and “e” will be compared. If the text handwritten on the layer 56 of the electronic medical record image 103 is a Chinese word of “disease”, the text will be compared word-by-word. If the character handwritten on the layer 56 of the electronic medical record image 103 is “123.4”, the text “1”, “2”, “3”, “.”, and “4” will be compared.

The electronic medical record system 40 can include a search module 404. The search module obtains the textual meaning and/or the representation of the texts on the layer 56 of the electronic medical record image 103 using the analysis module 403, and conduct a search according a search criterion on content of the layer 56 of the electronic medical record image 103 which match the search criterion. The searched result is displayed in a list in a sorted order. The search module 404 can also conduct a search on the database 201, 202 to search for the layer 56 of the electronic medical record image 103 in the database 201, 202 that matches the search criterion. The search result is displayed in a list in a sorted order. The search criterion can be based on at least one text, at least one number, or a combination of at least one text and at least one number. The search result can be sorted by date, medical record numbers, patients' birthdates, names, classifications of the medical record, or relevance to the search criterion.

In an embodiment of the present disclosure, the electronic medical record image 103 or the electronic medical record 34 may be stored in a format that is machine-readable and editable by the electronic medical record system 40 or in a portable document format (PDF) format which can be further secured and/or protected by a password. The medical personnel 16-18 can transmit the electronic medical record image 103 or the PDF file of the electronic medical record 34 to the patient 15 via the network communication 19. In an embodiment of the present disclosure, after the physician 17 performs a diagnosis, the physician 17 handwrites a text, inputs a text, draws a picture or a graph, or pastes a digital media file 53 on the layer 56 on the electronic medical record image 103 of the patient 15 (for example, an X-ray image, an ultrasound image, etc.) The physician 17 can annotate the digital media file 53, and the medical information can be copied and pasted to a referral form or an insurance reimbursement form which is saved as a PDF file. The PDF file is transmitted to the second handheld device 11 of the patient 15, thereby saving time required for rewriting the content of the traditional paper medical record and the traditional electronic medical record system on the referral form or the insurance declaration, and omitting additional steps of burning the digital media file 53 of the traditional paper medical record to a compact disc (CD). Therefore, the device 10 and the method for establishing an electronic medical record of the present disclosure save unnecessary expenditures for the patient and allow more money to be applied to treatment.

According to the device 10 and the method for establishing an electronic medical record of the present disclosure, the patient 15 can also directly sign on the electronic medical record image 103 (for example, a medical record exchange consent form, an anesthesia consent form, a surgical consent form, a donation consent form, or a power of attorney, etc.) by the device 10, so that the signed electronic medical record image 103 is directly stored in the electronic medical record 34. Therefore, time and processes of printing the electronic medical record image 103 for the patient 15 to sign on the electronic medical record image 103, scanning the electronic medical record image 103, and filing the electronic medical record image 103 are omitted. When the patient 15 signs on the device 10, the device 10 of the present disclosure can also be connected to a printer via the network communication 19 and directly transmit the signed consent form of the electronic medical record image 103 to the printer to print a copy for the patient 15. The signed consent form of the electronic medical record image 103 can also be directly transmitted to the second handheld device 11 of the patient 15 via the network communication 19. The transmission of the signed consent form can be encrypted by the encryption module 402 to generate a code and transmitted to the second handheld device 11 of the patient 15 which decodes the code and to retrieve the copy of the signed consent form of the electronic medical record image 103.

The electronic medical record system 40 can include a mobile medical record database 109. The electronic medical record image 103 is placed in one of the classifications according to a sorting criterion. The sorting criterion may be based on the appointment time of the patient 15 (for example, morning appointment, afternoon appointment, and night appointment), illness severity of the patient 15 (for example, general and severe), type of patient 15 (for example, general and baby), or types electronic medical record image 103 (for example, willingness form, medical record, and report). The mobile medical record database 109 can connect to the server 20 to access the electronic medical record 34 in the server 20 and upload the edited electronic medical record 34 so as to synchronously update the electronic medical record 34 stored in the server 20. As shown in FIG. 3, the mobile medical record database 109 includes a plurality of classifications 60-66 including willingness forms 60, today's appointments 61, first-time medical records 62, medical record library 63, babies 64, reports 65, others 66, etc. The others 66 can be temporary storage, ward records, or other classifications. The medical personnel 16-18 may establish the desired classification and subclassification thereof according to requirements. The temporary storage temporarily stores the deleted electronic medical record image 103. The ward records stores record related to the wards. Among all, each classification 60-66 may include a plurality of subclassifications 601, 611-613, 631-634, 641-642, and 651-652.

The willingness forms 60 may include a donation willingness form 601, etc. and stores willingness forms of the patients 15 to donate organs or tissues (for example, sperm, eggs, etc.)

Today's appointments 61 can include subclassifications of morning appointments 611, afternoon appointments 612, and night appointments 613, etc. The medical personnel can select the entire electronic medical record 34 or one electronic medical record image 103 of each of the patient who booked an appointment for today from the medical record library 63 and classifies the entire electronic medical record 34 or one electronic medical record image 103 into the corresponding classification in a sorted order. For example, the entire electronic medical record 34 or one electronic medical record image 103 of each of the patient who has booked an appointment at 9 am to 12 pm is classified into the morning appointments 611. The entire electronic medical record 34 or one electronic medical record image 103 of each of the patient who has booked an appointment at 12 pm to 6 pm is classified into the afternoon appointments 612. The entire electronic medical record 34 or one electronic medical record image 103 of each of the patient who has booked an appointment at 7 pm to 10 pm is classified into the night appointments 613. The sorting criterion may be appointment time, registration order, medical record number, birthdate of the patient, name, and the classification of the medical record.

The first-time medical record 62 stores the electronic medical record 34 of the first-time patient 15.

The medical record library 63 can include subclassifications of general 631, severe 632, postpartum confinement 633, and assistance 634, etc. The medical record library 63 stores the electronic medical records 34 of all patients, and is classified according to the disease degree, so that the medical personnel 16-18 can quickly find the corresponding electronic medical record 34. The general patient is classified into the general 631. The patient with a serious disease is classified into the severe 632. The patient who undergoes postpartum confinement is classified into the postpartum confinement 633. The patient who needs pregnancy assistance is classified into an assistance 634. The medical record library 63 can be connected to another server to back up all electronic medical records 34 in the medical record library 63 so as to further protect the electronic medical records 34 of the patients 15 and improve the reliability of the method and the device 10 for establishing an electronic medical record.

The babies 64 can include subclassifications of babies from postpartum confinement 641 and babies from wards 642, etc. The babies from postpartum confinement 641 stores the electronic medical record 34, birth records, reports of various examination results of the babies who stay in a general nursery. The babies from wards 642 stores the medical records, birth records, reports of various examination results of babies who stay in wards.

The reports 65 can include subclassifications of examination reports 651, pathological reports 652, etc. The examination reports 651 stores reports of various examinations. The medical personnel (for example, a nurse 16 or a medical technologist) can access an examination report of the patient 15 in the reports 65, print the examination report, and provide the examination report to the patient 15. Alternatively, the medical personnel can generate a corresponding code of the examination report (for example, QR-code) by the encryption module 402 of the electronic medical record system 40. The code is transmitted to the second handheld device 11 of the patient 15 by a handheld device 12-14 and is decrypted by the second handheld device 11 of the patient 15 to read the electronic medical record image 103.

The device 10 for establishing a medical record according to an embodiment of the present disclosure, the mobile medical record database 109 is stored in the storage device 107 of the device 10 and the medical record database 202 on the server 20. The medical personnel 16-18 can open the mobile medical record database 109 by the handheld device 11-14 to select an electronic medical record image 103 and place the selected electronic medical record image 103 in one of the classifications of the mobile medical record database according to a sorting criterion. The sorting criterion may be the time for the appointment of the patient 15, illness severity of the patient 15, types of the patient 15, or types of the electronic medical record image 103. According to an embodiment of the present disclosure, if the patient 15 has made an appointment in the afternoon, a nurse 16 searches for the electronic medical record 34 of the patient 15 from the medical record library 63 by the search module 404 of the device 10 for establishing electronic medical record according to the present disclosure. The electronic medical record 34 of the patient 15 is then moved from the medical record library 63 to the afternoon appointment 612 of the today's appointments 61 according to the appointment time of the patient 15. The physician 17 retrieve the electronic medical record 34 of the patient 15 from the afternoon appointments 612 of the today appointments 61 via the device 10 and inputs at least one text 50, handwritten at least one text (see 51 of FIG. 6), Draw at least one pattern (see 52 in FIG. 6), or annotate at least one digital media file (see 53 in FIG. 6) by the handwriting module 401 to record the condition of the patient 15. After having a medical consultation, the nurse 16 will move the electronic medical record 34 of the patient back to the medical record library 63.

The mobile medical record database 109 of the present disclosure enable the user to move the electronic medical record 34 of the patient 15 to a specific classification according to a sorting criterion, so that the relevant medical personnel 16-18 can administer the number of the patients in each classification or the condition of special patients. Therefore, on one hand, the medical personnel who is responsible for the relevant matters of the classification 16-18 can easily obtain real-time information of the classification, for example, how many patients are waiting for medical consultations, surgery, examination, or drug dispensing, etc. On the other hand, effectively managing the authorization of the medical personnel 16-18 to the electronic medical record 34, the medical personnel 16-18 can only access the electronic medical record 34 of the patients 15 in the department to which they are responsible for. For example, the physician 17 who is responsible for today's afternoon consultations can only access the electronic medical record 34 of the patients at the afternoon appointments 612. The medical technologist who is responsible for examination can only access the electronic medical record 34 of the patients who are waiting for the examination. Therefore, the mobile medical record database 109 of the present disclosure avoids the problem that the authorization of the medical personnel is too board, protects the privacy of the patients, and avoids the problem the medical personnel mistakenly edit the electronic medical records 34 of a wrong patient or tamper with electronic medical records 34.

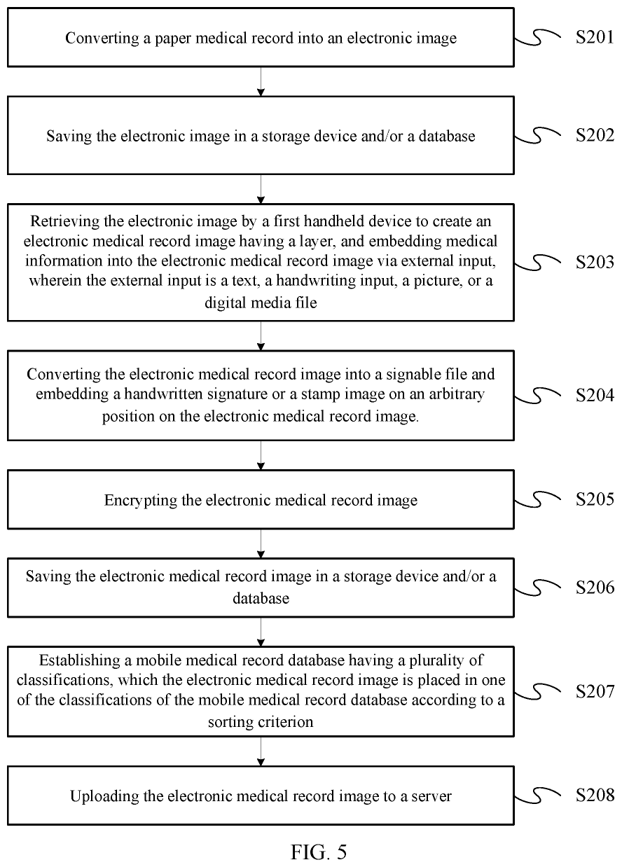

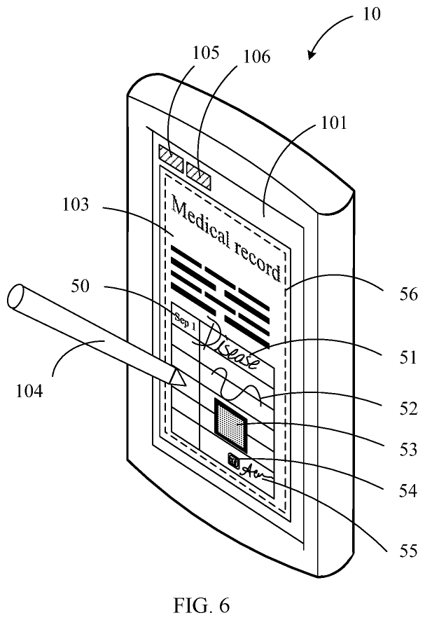

FIG. 4 is a flow chart of a method for establishing an electronic medical record according to an embodiment of the present disclosure. FIG. 5 is a flow chart of another method of establishing an electronic medical record according to an embodiment of the present disclosure. As shown in FIG. 4, the present disclosure provides a method of establishing an electronic medical record, comprising:

a step of S101 of converting a paper medical record 31 into an electronic image 32, which the paper medical record 31 can have one or more than one page and each page of the paper medical record 31 is converted into an electronic image 32;

a step of S102 of saving the electronic image 32 in a storage device 107 and/or a database 201, 202;

a step of S103 of retrieving the electronic image 32 by a first handheld device 12 to create an electronic medical record image 103 having a layer 56, and embedding medical information into the electronic medical record image 103 via external input, wherein the external input is a text (see 50 in FIG. 6), a handwriting input (see 51 in FIG. 6), a picture (see 52 in FIG. 6), or a digital media file (see 54 in FIG. 6);

a step of S104 of saving the electronic medical record image 103 in a storage device 107 and/or a database 201, 202.

In the step of S103, the digital media file 53 can be one of an image, a video, a recording, etc. The digital media file can be captured by the image capturing device 102 of the device 10 for establishing an electronic medical record. The image can be obtained by medical imaging, such as an ultrasound image or an X-ray image. The video can be obtained by medical imaging, such as an ultrasonography or an X-ray radiography. The recording can be recorded by a recording device of the device 10 for establishing an electronic medical record. The digital media file 53 can be played directly on the electronic medical record image 103 or played by a media player software installed in the device 10 for establishing an electronic medical record.

As shown in FIG. 5, the present disclosure provides another method of establishing an electronic medical record, comprising:

a step of S201 of converting a paper medical record 31 into an electronic image 32, which the paper medical record 31 can have one or more than one page and each page of the paper medical record 31 is converted into an electronic image 32;

a step of S202 of saving the electronic image 32 in a storage device 107 and/or a database 201, 202;

a step of S203 of retrieving the electronic image 32 by a first handheld device 12 to create an electronic medical record image 103 having a layer 56, and embedding medical information into the electronic medical record image 103 via external input, wherein the external input is a text (see 50 in FIG. 6), a handwriting input (see 51 in FIG. 6), a picture (see 52 in FIG. 6), or a digital media file (see 54 in FIG. 6);

a step of S204 of converting the electronic medical record image 103 into a signable file and embedding a handwritten signature (see 55 of FIG. 6) or a stamp image (see 54 of FIG. 6) on an arbitrary position into the electronic medical record image 103;

a step of S205 of encrypting the electronic medical record image 103;

a step of S206 of saving the electronic medical record image 103 in a storage device 107 and/or a database 201, 202

a step of S207 of establishing a mobile medical record database 109 having a plurality of classifications 60-66, which the electronic medical record image 103 is placed in one of the classifications 60-66 of the mobile medical record database according to a sorting criterion; and

a step of S208 of uploading the electronic medical record image 103 to a server 20.

In the step of S203, the digital media file 53 can be one of an image, a video, a recording, etc. The digital media file can be captured by the image capturing device 102 of the device 10 for establishing an electronic medical record. The image can be obtained by medical imaging, such as an ultrasound image or an X-ray image. The video can be obtained by medical imaging, such as an ultrasonography or an X-ray radiography. The recording can be recorded by a recording device of the device 10 for establishing an electronic medical record. The digital media file 53 can be played directly on the electronic medical record image 103 or on a media player software installed in the device 10 for establishing an electronic medical record.

In the method for establishing an electronic medical record according to an embodiment of the present disclosure, the method further includes generating a code encrypting the electronic medical record image 103, and transmitting the code to a second handheld device 11, decrypting the code by the second handheld device 11, and reads the electronic medical record image 103.

In the method for establishing an electronic medical record according to an embodiment of the present disclosure, the method further includes connecting to a printer via the network communication 19 to print the electronic medical record image 103. When being reviewed by an authority (for example, the national health insurance administration), the entire electronic medical record 34 and all relevant medical records in the entire database can be printed by the method for establishing an electronic medical record of the present disclosure, so that all the electronic medical records 34 can be immediately printed and submitted to the authority.

Please refer to FIG. 6 and FIG. 7 which are perspective views of a device for establishing an electronic medical record from different angles according to an embodiment of the present disclosure. The device 10 is provided with an image capturing device 102, a touch panel 101, an electronic medical record system 40, a storage device 107, and a network communication device 108. The image capture device 102 converts a paper medical record 31 into an electronic image 32. In an embodiment of the present disclosure, the paper medical record 31 can have one or more than one page. Each page of the paper medical record 31 is converted into an electronic image 32. The electronic image 32 of the paper medical record is saved in the same storage path to establish the electronic medical record 34 of the patient. The storage path can connect to the server 20, the template database 201 on the server 20, the medical record database 202 on the server 20, or the storage device 107 of the device 10. The electronic medical record system 34 can be manually named or automatically named by the electronic medical record system 40. The name of the electronic medical record 34 can be based on the patient's identity card number, uniform ID number, name, birthdate, etc. The electronic medical record system 40 can automatically generate a sequential serial number or a random serial number.

The image capturing device 102 can be a camera that can take a picture of the paper medical record 31 to obtain the electronic image 32. The image capture device 102 can be connected to a software or a mobile application to adjust contrast, brightness, and angle, or alignment to edges of the paper medical record 31 to increase the clarity of the electronic image 32.

The touch panel 101 can be a resistive touch panel, a capacitive touch panel, or an acoustic wave touch panel. The touch panel 101 receives a touch signal and a data input signal from a stylus 104 or a finger. If the touch panel 101 is a resistive touch panel, the stylus 104 or a finger presses the panel with an external force so that an upper electrode layer is brought into contact with a lower electrode layer which results in a voltage changes and generates the touch signal. If the touch panel 101 is a capacitive touch panel, the stylus 104 or the finger changes capacitance of the panel which can be used to deduct the location of the touch event and generate the touch signal. If the touch panel 101 is an acoustic wave touch panel, the surrounding emitters and receivers form a mesh to detect the signal intercepted by the stylus 104 or the finger and generate the touch signal. The data input signal can be derived from a handwriting input or pressing a keyboard of the touch panel 101.

The electronic medical record system 40 converts the electronic image 32 into an electronic medical record image 103 having at least one layer 56. Therefore, a user 15-18 can input a text 50 (and/or a handwritten text 51), draw a picture 52, or paste a digital media file 53 directly onto an arbitrary position of the electronic medical record image 103 or an arbitrary position of the layer 56. The electronic medical record system 40 can be a built-in system, a mobile application (app), or a software. The electronic medical record system 40 can be installed as a built-in system in the device 10 for establishing an electronic medical record or in the handheld device 11-14, or installed as a mobile application in the handheld device 11-14. In an embodiment of the present disclosure, when a user 15-18 is inputting medical information onto the electronic medical record image 103 and/or the layer of the electronic medical record image 103 (for example, handwriting, drawing, or pasting a digital media file), the handwriting module 401 can perform edition on the existing layer 56 or adding at least one layer 56 on the electronic medical record image 103. The user 15-18 can also add at least one layer 56 according to actual needs to further handwrite a text 51, draw a picture 52, or a digital media file 53, for example, the handwritten text 51 is on a first layer, the input text 50 is on a second layer, the picture 52 is on a third layer, and the digital media file 53 is on a fourth layer. Therefore, each content is located on a separate layer and the content of each of the layers can be individually modified or moved, for example, moving one of the layers to another location. In addition, the layers can be overlapped with each other. For example, the user 15-18 can move the handwritten text 51 of the first layer to the top of the digital media file 53 of the fourth layer or add a fifth layer having another handwritten text on the digital media file 53 of the fourth layer, so as to annotate to the digital media file 53 and more comprehensively describe the condition of the patient 15. In an embodiment of the present disclosure, because the layer 56 is positioned on the electronic image 32, the electronic image 32 serves as a background and is not editable. The layer 56 can be in the form of a virtual paper. The user 15-18 can further edit the layer 56. The layer 56 can be transparent or other colors. A size of the layer 56 can be the same or substantially the same as a size of the electronic image 32. A size of the layer 56 can also be the same or substantially the same as a size of the input text 50, the handwritten text 51, the picture 52, or the digital media file 53. In an embodiment of the present disclosure, the layer 56 can be a text box, a picture box, a drawing canvas, or a digital media file box. The layer 56 can be moved to another location of the electronic medical record image 103 by dragging the electronic medical record image 103. When handwriting a text 51 or drawing a picture 52 on the electronic medical record image 103, the text 51 or the picture 52 can be set as non-editable to prevent being moved or tampered. In the device 10 and the method for establishing an electronic medical record of the present disclosure, the user 15-18 can further adjust the size of the digital media file 53. The user 15-18 can also zoom out the digital media file 53 to save the space of the electronic medical record image 103. The digital media file 53 can be zoomed in when viewing the digital media file 53 to view the digital media file 53 more clearly.

The user 15-18 can also use the handwriting module 401 of the electronic medical record system 40 to sign the electronic medical record image 103, so that the user 15-18 can handwrite a signature 55 or paste a stamp image 54 at an arbitrary position on the electronic medical record image 103 which is served as a virtual paper. In an embodiment of the present disclosure, the user (for example, the physician 17) writes a diagnosis on the electronic medical record image 103 of the patient 15 and then signs a signature 55 on the electronic medical record image 103 and/or pastes a stamp image 54 to add a stamp thereon. The stamp image 54 can be secured by a password, a fingerprint recognition, a face recognition, or a pupil recognition so that only the user himself 17 can access the stamp image 54. The stamp image 54 can also be stored in an account so that the user 17 accesses the stamp image 54 thereof according to the authorization. In an embodiment of the present disclosure, the electronic medical record image 103 can be a consent form or a power of attorney, for example, a medical record exchange consent form, an anesthesia consent book, a surgical consent form, a donation consent form, a power of attorney for collecting medical record, or a power of attorney for collecting medication. The patient 15 can sign a signature 55 or paste a stamp image 54 on the consent form or the power of attorney.

The electronic medical record system 40 can display at least one button 105, 106 which receives an instruction from a stylus 104 or a finger and then executes the instruction. A first button 105 is functional for adding a page. When the stylus 104 or the finger clicks the first button 105, a new page is added. The first button 105 is further functional for determining the type of the page can be added in a currently displayed interface of the electronic medical record system 40. If the currently displayed interface is a list of the electronic medical record images 103 in the classifications 60-66 or subclassifications 601, 611-613, 641-642 thereof on a main menu, the first button 105 is functional for adding a new electronic medical record 34 in the classification 60-66 or subclassifications 601, 611-613, 641-642 thereof. The electronic medical record 34 can contain at least one electronic medical record image 103. If the currently displayed interface is the electronic medical record image 103 of the electronic medical record 34, the first button 105 is functional for adding a new electronic medical record image 103 in front of or behind the electronic medical record image 103. The new electronic medical record image 103 can be a medical record form, a medical record continuation page, an examination form, a consent form, a prescription, a blood drawing form, a medical examination form, an arrears form, a reminder list, or a short-stay observation form. In an embodiment of the present disclosure, the medical personnel 16-18 may add a reminder list on the front page of the electronic medical record 34 of the patient 15 to track the unfinished items of the patient 15, for example, regular blood test, remaining examination items to be carried out, etc.

A second button 106 can be functional for displaying options of the classifications 60-66 of the mobile medical record database 109. When the stylus 104 or the finger clicks the second button 106, the touch panel 101 displays the options of the classifications 60-66 of the mobile medical record database 109. When one of the classifications 60-66 is clicked, the options of the subclassification 601, 611-613, 641-642, and 651-652 is displayed. A user 15-18 can click on the classifications 60-66 or subclassifications 601, 611-613, 641-642 of the mobile medical record database 109 to view a list of the electronic medical record images 103 of each of the classifications 60-66 or subclassifications 601, 611-613, 641-642.

The present disclosure provides a device and a method for establishing an electronic medical record so that medical personnel can directly record, draw, and even paste various types of digital media files onto an electronic medical record by a handheld device. The device and the method for establishing an electronic medical record of the present disclosure are not limited by standardized menus and text fields that are provided by the traditional electronic medical record system. Each patient's condition can be accurately recorded to facilitate tracking of the disease.

The device for establishing the electronic medical record directly read the text that is handwritten by medical personnel on an electronic medical record so that the handwriting of the medical personnel can be preserved in the electronic medical record. Therefore, each electronic medical record can be traced, signature verification and handwriting identification are allowed, thereby solving the problem of the traditional electronic medical record system that it is still impossible to distinguish the identity of the user, and avoiding the problem of the traditional electronic medical record system that a person who has bad intentions can commit an impersonation using this loophole. The patient's rights and interests are thus protected. The present disclosure also solves the problems that the patient cannot directly sign a consent form on the traditional electronic medical record system and save large quantities of paper. Furthermore, the handwritten text and the input text on the electronic medical record of the present disclosure is machine-readable and can be used to perform functions, such as searching. Therefore, the device for establishing an electronic medical record of the present disclosure solves the disadvantage that the traditional paper medical records are difficult to find. It also solves a loophole of the traditional electronic medical record system that actual identify of the writer cannot be identified and improves credibility and practicability of the electronic medical record.

The present disclosure directly captures the electronic image of the paper medical record to create an electronic medical record image having a layer thereon, such that the electronic medical record image serves as a virtual paper and allows the medical personnel to write directly on the electronic medical record image and establishes the electronic medical record, without learning a manual of a new electronic medical record system and spending time to adjust to the new system. Therefore, the present disclosure avoids the problem that the medical personnel fail to properly record the patient's condition because they do not know how to operate or are unfamiliar with the system.

Although the present invention has been described with preferred embodiments thereof, the above preferred embodiments is not used to limit the present invention. One of ordinarily skill in the art can carry out changes and modifications to the described embodiment without departing from the scope and the spirit of the invention that is intended to be limited only by the appended claims.

1. A method of establishing an electronic medical record, comprising steps of:
converting a paper medical record into an electronic image;
saving the electronic image in a database;
retrieving the electronic image by a first handheld device from the database to create an electronic medical record image having a layer thereon, and embedding medical information into the electronic medical record image via external input, wherein the external input is a text, a handwriting input, a picture, or a digital media file; and
saving the electronic medical record image back to the database.

2. The method of establishing the electronic medical record according to claim 1, wherein the database is located in a server.

3. The method of establishing the electronic medical record according to claim 1, wherein the database is located in a storage device of the first handheld device.

4. The method of establishing the electronic medical record according to claim 1, wherein the method further comprises adding another layer on the layer of the electronic medical record image by the user.

5. The method of establishing the electronic medical record according to claim 1, wherein the method further comprises recognizing the text when the text is handwritten on the electronic medical record image, such that the first handheld device can directly read the text that is handwritten on the electronic medical record image.

6. The method of establishing the electronic medical record according to claim 5, wherein the method further comprises conducting a search according to the text in the electronic medical record image by a search criterion and displaying a searched result in a list in a sorted order.

7. The method of establishing the electronic medical record according to claim 1, wherein the method further comprises saving an original content of the electronic medical record image while modifying medical information thereof.

8. The method of establishing the electronic medical record according to claim 1, wherein the method further comprises establishing a mobile medical record database, establishing a plurality of classifications in the mobile medical record database, classifying the electronic medical record image, and placing the electronic medical record image in one of the classifications of the mobile medical record database according to an order of classification.

9. The method of establishing the electronic medical record according to claim 1, wherein the method further comprises converting the electronic medical record image into a signable file and embedding a handwritten signature or a stamp image on an arbitrary position on the electronic medical record image.

10. The method of establishing the electronic medical record according to claim 1, wherein the method further comprises encrypting the electronic medical record image and accessing the electronic medical record image according to an authorization of the user.

11. The method of establishing the electronic medical record according to claim 1, wherein the method further comprises encrypting the electronic medical record image, transmitting the encrypted file to a second handheld device, decrypting the encrypted file by the second handheld device, and reading the electronic medical record image by the second handheld device from the decrypted file.

12. A device for establishing an electronic medical record, comprising:
an image capturing device, wherein the image capturing device is configured to convert a paper medical record into an electronic image;
a touch panel, wherein the touch panel is configured to receive a touch signal and a data input signal, and to display the electronic image;
an electronic medical record system, wherein the electronic medical record system is configured to retrieve the electronic image from the database to create an electronic medical record image having a layer thereon and is configured to embed medical information into the electronic medical record image via external input, wherein the external input is a text, a handwriting input, a picture, or a digital media file;
a storage device for saving the electronic medical record image; and
a network communication device connected to a server for uploading the electronic medical record image to the server and accessing the electronic medical record image in the server.

13. The device for establishing the electronic medical record according to claim 12, wherein the server comprises:
a template database having an electronic image of a paper medical record template, wherein the electronic medical record system accesses the paper medical record template from the template database via the network communication device; and
a medical record database having the electronic medical record image, wherein the medical record database is connected to the storage device via the network communication device and stores the electronic medical record image that is saved in the storage device.

14. The device for establishing the electronic medical record according to claim 12, wherein the device further comprises a template database having an electronic image of a paper medical record template.

15. The device for establishing the electronic medical record according to claim 12, wherein the electronic medical record system comprises an encryption module for encrypting the electronic medical record image, whereby the user can access the electronic medical record image according to an authorization of the user.

16. The device for establishing the electronic medical record according to claim 12, wherein the electronic medical record system comprises an analysis module, wherein the analysis module is configured to recognize the text when the text is handwritten on the electronic medical record image, such that the electronic medical record system can directly read the text that is handwritten on the electronic medical record image.

17. The device for establishing the electronic medical record according to claim 16, wherein the electronic medical record system comprises a search module for conducting a search according to the text in the electronic medical record image by a search criterion and displaying a searched result in a list in a sorted order.

18. The device for establishing the electronic medical record according to claim 12, wherein the electronic medical record system comprises a mobile medical record database, wherein the mobile medical record database comprising a plurality of classifications and the electronic medical record image is placed in one of the classifications of the mobile medical record database according to a sorting criterion.
